(19) United States
(12) Patent Application Publication
(10) Pub. No.: US 2009/0259257 A1
(43) Pub. Date: Oct. 15, 2009

(54) Pedicule-Based Motion- Preserving Device

(75) Inventor: Julien J. Prevost, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: WARSAW ORTHOPEDIC, INC., Warsaw, IN (US)

(21) Appl. No.: 12/103,417

(22) Filed: Apr. 15, 2008

(51) Int. Cl. A61B 17/70

(52) U.S. Cl. 606/255; 606/250; 606/252

(57) ABSTRACT

Methods and devices for a motion-preserving spinal rod are disclosed including an elongate first rod portion extending generally along a first curved path wherein at least a portion of the first curved path substantially approximates a kinematic curve defined by flexion and extension of a superior vertebra relative to an inferior vertebra. An elongate second rod portion is coupled to the first rod portion. It extends along a second curved path wherein at least a portion of the second curved path substantially approximates a posterior lordotic curve, and wherein the first curved path is oriented relative to the second curved path to substantially form an S-shaped curve. A core extends between the first rod portion and the second rod portion and a resilient damper is disposed about the core.

### FIELD OF THE INVENTION

The present disclosure relates to devices and methods for preserving motion between vertebrae, and more particularly, to a device and method for improving posterior spinal function with a pedicle-based implant.

### BACKGROUND

Severe back pain, limited motion, and nerve damage may be caused by injured, degraded, or diseased spinal anatomy. Affected spinal joints, and particularly discs and ligaments, can be difficult to treat externally and may necessitate surgery.

In some instances, the diseases, injuries or malformations affecting spinal motion segments are treated by fusing two adjacent vertebrae together using transplanted bone tissue, an artificial fusion component, or other compositions or devices. In some surgical treatments, posterior rods may be attached to variously affected spinal levels to inhibit or limit motion, with or without, spinal fusion. These posterior rods are frequently rigid rods which substantially, if not totally, eliminate freedom of motion for bending in flexion and extension. Other important motions may similarly be eliminated. Therefore, alternatives to substantially rigid rod systems are needed which allow for certain motion and which more closely approximate the natural function of the motion segments.

### SUMMARY

This disclosure offers an improved device and method for preserving motion with a pedicle-based dynamic rod. According to one embodiment, a motion-preserving spinal rod is disclosed comprising an elongate first rod portion extending generally along a first curved path. The first rod portion has a distal end, a proximal end and an intermediate portion extending therebetween. At least a portion of the first curved path substantially approximates a kinematic curve defined by flexion and extension of a superior vertebra relative to an inferior vertebra. An elongate second rod portion is coupled to the first rod portion and extends along a second curved path. The second rod portion includes a distal end, a proximal end and an intermediate portion extending therebetween. At least a portion of the second curved path substantially approximates a posterior lordotic curve. The first curved path is oriented relative to the second curved path to substantially form an S-shaped curve with the second curved path. A core extends between the first rod portion and the second rod portion and a resilient damper is disposed about the core between the first and second rod portions. The resilient damper is configured to provide resilient dampening of compressive force during vertebral extension.

In another aspect, a motion-preserving spinal rod is disclosed comprising the elongate first rod portion and the elongate second rod portion coupled to substantially form an S-shaped curve with the second curved path. A core extends between the first rod portion and the second rod portion with a damper disposed about the core, between the first and second rod portions. The damper is configured to provide dampening of compressive force during vertebral extension. A sheath has first and second ends attached to the first rod portion and the second rod portion. The sheath substantially surrounds the variable stiffness damper. The sheath is configured to provide resilient dampening of tensile force during vertebral flexion and limitation of vertebral motion during flexion.

In some embodiments, a motion-preserving spinal rod is disclosed comprising a generally S-shaped, elongate rod. The rod comprises a stem portion having at least one first diameter, and extends longitudinally from a base portion having at least one second diameter larger than the first diameter. The stem portion extends at least partially along a first curved path, substantially approximating a kinematic curve generated by flexion and extension of adjacent superior and inferior vertebrae. The base portion extends at least partially along a second curved path. The second curved path substantially approximates a posterior lordotic curve. The first curved path is oriented relative to the second curved path to substantially form an S-shaped curve with the second curved path. A collar is slidingly disposed around the stem portion, the collar having a first end and a second end, and being adapted to interface with a vertebral anchor. A first resilient damper is disposed about the stem portion and positioned between the base portion first end and the collar second end. It is configured to provide resilient dampening of compressive force exerted by the collar during vertebral extension. The base portion first end is configured to limit movement of the first resilient damper during vertebral extension and a retention member coupled to the stem portion first end.

In another exemplary aspect, a method of stabilizing a spinal motion segment with a motion-preserving spinal rod includes securing a first anchor to a first vertebra and securing a second anchor to a second vertebra. The method also includes selecting a motion-preserving spinal rod, wherein the motion-preserving spinal rod comprises an elongate first rod portion extending generally along a first curved path substantially approximating a kinematic curve defined by flexion and extension of a superior vertebra relative to an inferior vertebra. The rod also comprises an elongate second rod portion coupled to the first rod portion, the second rod portion extending generally along a second curved path substantially approximating a posterior lordotic curve. The first curved path is oriented relative to the second curved path to substantially form an s-shaped curve with the second curved path. A core extends between the first rod portion and the second rod portion, and a resilient damper is disposed about the core, between the first and second rod portions. The resilient damper is configured to provide resilient dampening of compressive force during vertebral extension, positioning the motion-preserving spinal rod between the first and second anchors, and securing the motion-preserving spinal rod to the first and second anchors.

These and other features will become apparent from the following description.

### BRIEF DESCRIPTION OF THE DRAWINGS

FIG. 1 is an isometric view of the device according to one exemplary embodiment installed on adjacent pedicles on adjacent vertebrae.

FIG. 2 is a side view of a device according to one exemplary embodiment.

FIG. 3 is an cross-section view of the exemplary device shown in FIG. 2.

FIG. 4 is a side view of the device according to one exemplary embodiment attached to adjacent vertebrae.

FIG. 5 is a side view of another device according to one exemplary embodiment.

FIG. 6 is an exemplary cross-section view of the device shown in FIG. 6.

FIG. 6a is an exemplary cross-section view of the device according to one exemplary embodiment.

FIGS. 7a-7c are lateral cross-sectional views according to alternative embodiments of a portion of the devices.

FIGS. 8a and 8b are longitudinal cross-sectional views of a damper according to various embodiments.

FIG. 9 is a lateral cross-sectional view of a damper according to one exemplary embodiment.

FIG. 10 is a side view of a device according to one exemplary embodiment.

FIG. 11 is an exemplary cross-section view of the device shown in FIG. 10.

FIG. 12 is a side view of the device according to one exemplary embodiment installed across three vertebrae.

### DETAILED DESCRIPTION

The present disclosure relates to devices and methods for preserving motion between vertebrae, and more particularly, to a device and method for improving posterior spinal function with pedicle-based implants. These pedicle-based implants allow for some motion, and may more closely approximate the natural function of the motion segments than prior devices.

For the purposes of promoting an understanding of the principles of the invention, reference will now be made to embodiments or examples illustrated in the drawings, and specific language will be used to describe the same. It will nevertheless be understood that no limitation of the scope of the invention is thereby intended. Any alteration and further modifications in the described embodiments, and any further applications of the principles of the invention as described herein are contemplated as would normally occur to one skilled in the art to which the disclosure relates.

Anatomical planes are referred to herein for the purpose of more clearly describing the disclosed embodiments. It is generally understood that the coronal plane bisects the body longitudinally in the medial-lateral direction. The sagittal plane is perpendicular to the coronal plane and bisects the body longitudinally in the anterior-posterior direction. The axial plane traverses the body laterally and is perpendicular to both the sagittal and coronal planes.

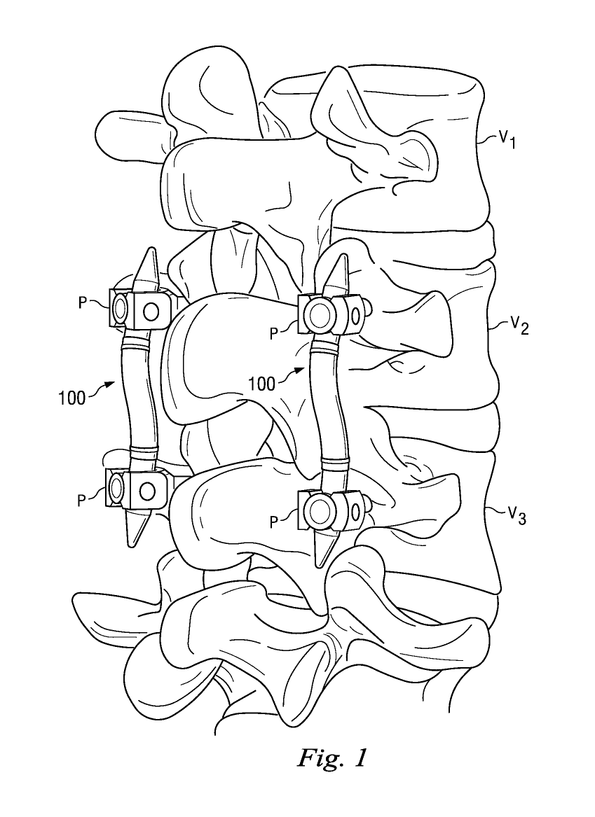

Referring to FIG. 1, exemplary embodiments of the proposed device 100 are shown installed along a representative section of the spine. A representative posterior isometric view of a section of the lumbar region of the spine is shown comprising vertebrae labeled V₁, V₂ and V₃. Pedicle screws P are shown attached through respective pedicle portions of vertebrae V₂ and V₃.

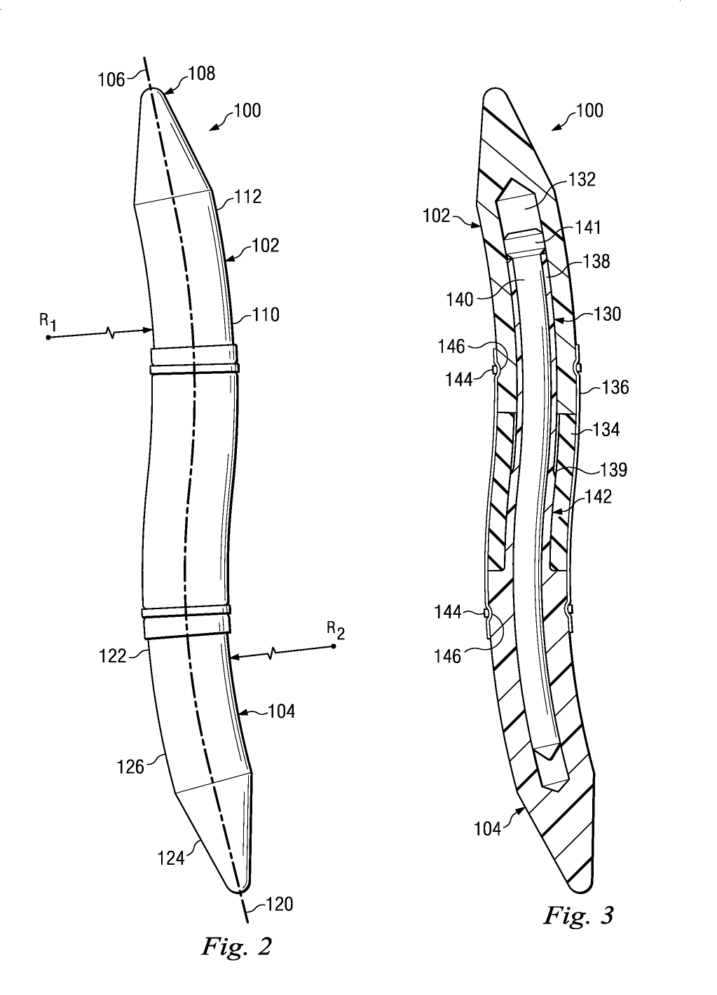

Turning now to FIGS. 2-4, an exemplary device 100 according to one embodiment is described. Device 100 shown in FIG. 2 extends generally along a longitudinal axis and consists generally of an elongate first rod portion 102 coupled to an elongate second rod portion 104. First rod portion 102 extends generally along a first curved path 106, and has a superior end 108 and an inferior end 110 with intermediate portion 112 extending therebetween. First curved path 106 substantially approximates a kinematic curve generated in the sagittal plane by flexion and extension of adjacent superior and inferior vertebrae, the kinematic curve having anterior concavity (or opening towards the front of the body).

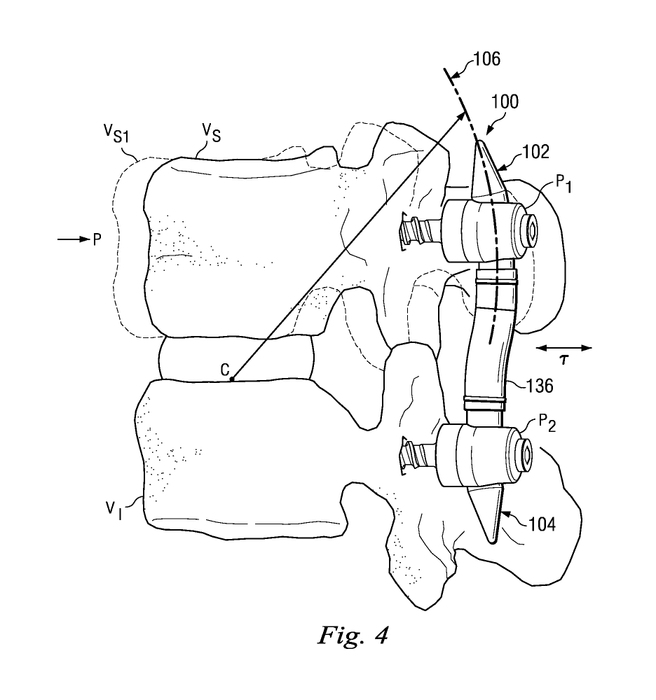

Turning briefly to FIG. 4, it is shown more particularly that first curved path 106 may be defined by the motion of a hypothetical point near a rod attachment location on a superior vertebra. The point follows an arcing motion defined by a motion segment center of rotation C as superior vertebra Vₛ rotates with respect to inferior vertebra Vᵢ from a first position in extension to a second position in flexion. Kinematic curvatures may have radii ranging from about 30 mm to about 55 mm, and may be based on the spinal level being treated. Therefore, in some embodiments a selection of three first rod portions 102 having respective curvatures with radii 30 mm, 45 mm, 50 mm and 55 mm may be provided to a surgeon. The surgeon then may chose which first rod portion 102 would best fit an individual. First curved path 106 may be pre-formed in the first rod portion 102 or added by a surgeon or technician before or during surgery.

Second rod portion 104 extends generally along a second curved path 120, and has a superior end 122 and an inferior end 124 with intermediate portion 126 extending therebetween. Second curved path 120 substantially approximates a lordotic curve of the lumbar spine.

The lordotic curve, or lordosis, is a curve in the sagittal plane with posterior concavity (concavity towards the back of the body). Normal lumbar lordosis is typically 30 to 50 degrees and is formed essentially by the five lumbar vertebrae L1-L5. A typical lordotic curvature may have a radius of 60 mm. Other arrangements and curvatures are contemplated, however, including rod portions having a curvature defined by larger or smaller radii. In some instances, custom lordotic curvatures are fitted directly to an individual patient.

As shown in FIG. 2, second curved path 120, follows the second rod portion and is concave posteriorly while first curved path 106 is concave anteriorly, resulting in a general S-curve when first and second rod portions 102 and 104 are coupled together. Either curved path 106 or 120 may be approximated using standard data before or during manufacturing, using patient specific data obtained via an x-ray or other scanning device before or during surgery, using patient specific data obtained externally by taking measurement along the surface of the body, using visual or measured data from trial-fitting during surgery, or by other measurement means known or developed in the art.

Although first rod portion 102 is shown with a somewhat tapered, or narrowed superior end 108, and second rod portion 104 is shown with a similarly tapered inferior end 124, it is contemplated that either end 108 or 124 may be shaped according to other embodiments, such as flat, rounded, sharply pointed, and the like. Having a tapered end 108 or 124, may enable device 100 to more easily pass through intervening tissue or other anatomy during surgery. Having a blunt end may provide a connection interface adapted for extending the length of device 100 or for attaching to other similar or different devices. A blunt end might further function to prevent tissue penetration or trauma.

Turning now to FIG. 3, a longitudinal, cross-section view of device 100 is shown extending generally along a longitudinal axis. Device 100 may include a core 130 extending from second rod portion 104 into a longitudinal channel 132 formed in first rod portion 102. A resilient damper 134 is shown surrounding core 130 and further located between first and second rod portions 102 and 104. A bio-compatible, flexible sheath 136 surrounds resilient damper 134 and is connected at either end to both first and second rod portions 102 and 104.

Core 130 may be comprised of a sleeve 138 and an internal rod 140, both extending generally between first and second rod portions 102 and 104. Sleeve 138 may be integrally formed with the second rod portion 104 or a separate, attached component. Internal rod 140 is generally disposed inside sleeve 138 and may add additional strength and functionality to device 100. Sleeve 138 may include a diameter reduction represented by a shoulder 139, which may be included to change the stiffness or bending properties of core 130 along the motion path. For example, core 130, sleeve 138 and internal rod 140 may be modified to provide more anterior-posterior translation of a motion segment during flexion.

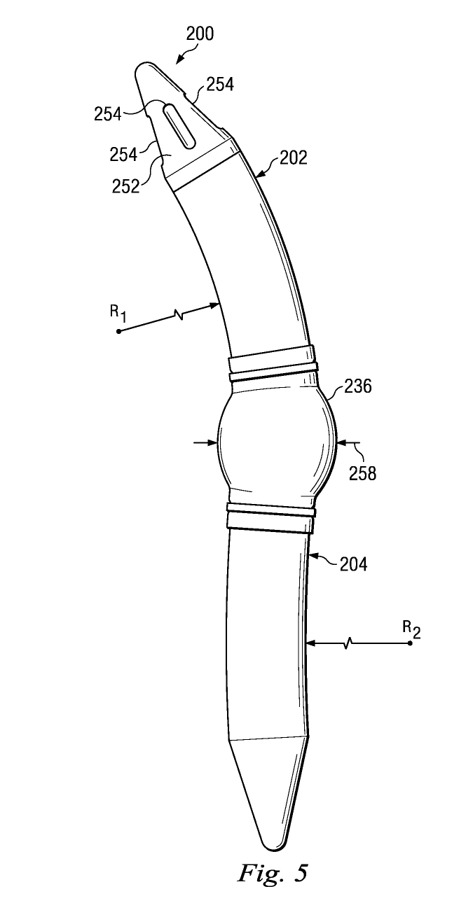

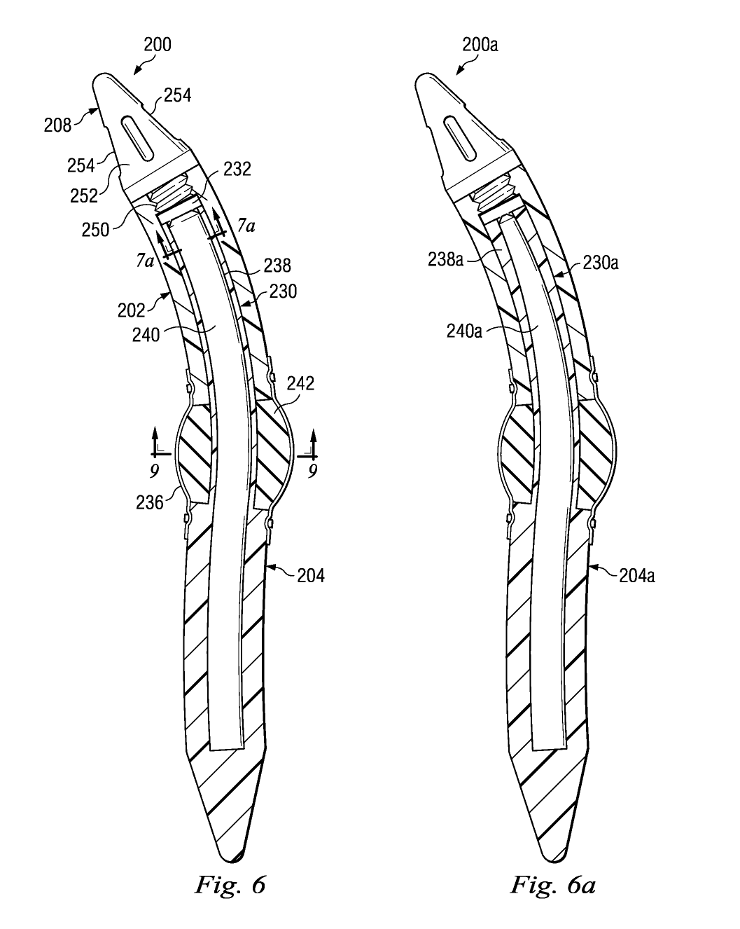

Internal rod 140 may also have an enlarged cap-head 141 designed for one or more of the following reasons: to function as a hard stop during compression of device 100 and to limit positioning of internal rod 140 with respect to sleeve 138. In other embodiments, an enlarged cap-head may provide for grasping or removal of an internal rod by a surgical tool. Internal rod 140 may be formed of a rigid material or a flexible material to provide desired properties, as explained with reference to FIGS. 5 and 6.

Since internal rod 140 follows first curved path 106, first rod portion 102 may slide along internal rod 140, which sliding will be further described below. Resilient damper 134 occupies an intermediate recess 142 between first and second rod portions 102 and 104. Resilient damper 134 provides for a resilient dampening between first and second rod portions 102 and 104 when a compression force is applied by the first rod portion 102 during vertebral extension.

Sheath 136 may be attached to first rod portion inferior end 110 and second rod portion superior end 122, as shown in FIG. 3. Sheath 136 surrounds resilient damper 134 and may be fixed to ends 110 and 122 by a circular band 144. In turn, circular band 144 may compress sheath 136 into ring groove 146 circumscribing ends 110 and 122. In other embodiments, sheath 136 may be crimped to first and second end portions 102 and 104, with or without accompanying features such as ring groove 146. In other embodiments, sheath 136 may be attached by other methods such as traditional plastics or metal welding, sonic welding, laser welding, crimping, gluing, stitching, and the like. Sheath 136 provides a travel limit to prohibit first and second rod portions 102 and 104 from sliding apart beyond a designed distance by providing a tension force. In this embodiment, sheath 136 may also provide resilient dampening in flexion.

Turning now to FIG. 4, a side view is shown of device 100 installed between two adjacent superior and inferior vertebrae Vₛ and Vᵢ. Device 100 has first rod portion 102 attached to a first pedicle screw P₁ threaded into superior vertebra Vs and second rod portion 104 attached to a second pedicle screw P₂ threaded into inferior vertebra Vᵢ. It is contemplated that device 100 may be compatible with anchors and pedicle screws from a variety of companies. One suitable pedicle screw design and method of installation is shown in published U.S. Patent Application 2005/0171540 (filed Dec. 10, 2003, incorporated herein by reference in its entirety, said application being commonly owned by assignee).

In some cases of deformity, such as spondylolisthesis, one or more vertebral bodies may be displaced with respect to each other. In such a deformity, it is desirable to reduce the extent of displacement, by re-positioning the displaced vertebral bodies. A spondylolisthesis reduction may be performed on one or more vertebra to restore spinal alignment in the sagittal plane, for example. Dislocations may include an anterior-posterior shift in the sagittal plane, a medial-lateral shift in the coronal plane, and shifts along multiple anatomical planes or between anatomical planes.

FIG. 4 shows superior and inferior vertebrae Vₛ and Vᵢ separated by an intervertebral disc D₁. Superior vertebra Vₛ—in solid lines, is represented in a dislocated position labeled Vₛ₁—in dashed lines. Vₛ is shifted in the anterior direction A. The shift directions are shown by arrow A-P, which in this example represents anterior to posterior movement in the sagittal plane. It is desired that the position of vertebra Vₛ₁ be corrected by moving vertebra Vₛ₁ in the posterior direction P to the position represented by Vₛ. In order to maintain vertebra Vₛ in the corrected position, pedicle screws P₁ and P₂ may be fitted with a device 100, according to one embodiment.

Since Vₛ will seek to return to its Vₛ₁ position, a shear stress τ (tau), represented by arrows τ, will act through a portion of the device along the axial plane. An additional shear stress will be placed on device 100 by the functional requirements normally placed on spinal motion segments. Thus, device 100, and in particular, core 130 are configured to resist anterior-posterior and medial-lateral shear forces between superior and inferior vertebrae Vₛ and Vᵢ while still allowing for some spinal bending and rotation.

As a description of spinal bending, the motion of first rod portion 102 sliding along first curved path 106, allows device 100 to preserve motion. The sliding interface between the first and second rod portions 102 and 104 extends along first curved path 106. When superior vertebra Vₛ rotates in flexion, first rod portion 102 pulls away from second rod portion 104 along first curved path 106 until resistance is met by sheath 136, or by a designed hard stop internal or external to device 100. As superior vertebra Vₛ rotates in extension, first rod portion 102 returns along first curved path 106 towards second rod portion 104 until its motion is restrained by compressing resilient damper 134 against second rod portion 104. In other embodiments the motion in extension may be limited by a hard stop.

Thus, device 100 provides for restriction of at least one type of undesirable motion (in this case, anterior-posterior shifting of Vₛ with respect to Vᵢ), while simultaneously providing for other relative movement between the adjacent vertebral bodies (flexion-extension bending between Vₛ and Vᵢ). This unique combination of functionality may help to maintain, or restore motion substantially similar to the normal bio-mechanical motion provided by a natural intervertebral disc and its associated facet joints.

Turning now to FIGS. 5 and 6, another exemplary embodiment of the device is shown. FIG. 5 is a perspective view of a device 200 according to one exemplary embodiment. Since, device 200 comprises many similar features as compared to device 100, described above, similar features will be referred by name but not fully described here. As shown in FIG. 5, exemplary device 200 has first and second rod portions 202 and 204. A spherical, or oval-shaped damper 242 is shown in cross-section in FIG. 6, located between first and second rod portions 202 and 204. Damper 242 is a laterally surrounded by a bio-compatible, flexible sheath 236.

In this embodiment, as first rod portion 202 pulls away from second rod portion 204 along kinematic curve 206, sheath 236 may offer resilient resistance in tension. As shown by arrows 258, sheath 236 compresses against resilient damper 242 when sheath 236 is tensed during flexion of the spine. Thus, by pressing against damper 242, sheath 236 may provide a resilient end resistance when the first rod portion 202 nears a determined travel limit for spinal bending in flexion.

FIG. 6 is an exemplary cross-section view of device 200. First rod portion 202 has a modified superior end 208. Superior end 208 comprises a threaded portion 250 extending from a cap portion 252. Thus, cap portion 252 is removable to expose a longitudinal channel 232 and an internal core 230. In addition, cap portion 252 may include tool interfaces 254 to provide secure engagement with a tool. In other embodiments, it is contemplated that cap portion 252 may be releasably attached or permanently attached by other methods such as, for example, sonic welding, gluing, snap-fitting, cam locking, slot or bayonet locking, and the like.

In this embodiment, core 230 comprises internal rod 240 which may be exchanged among various alternatives constructed from different materials. Alternatively constructed internal rods 240 may provide the option to change a flexible core to a more rigid core. Such an exchange may be performed during manufacturing or at a later time, such as before or during surgery. Alternatively, the internal rod 240 may be fixed, or permanently attached to second rod portion 204 and its accompanying sleeve 238. In another embodiment, internal rod 240, second rod portion 204 and sleeve 238 may be integrally formed into a monolith (see base portion 404 and stem portion 430 in FIG. 11). In yet another embodiment internal rod 240 may be removed, such as in a case for treating a simple stenosis, or to achieve more axial translation during motion, and particularly in flexion.

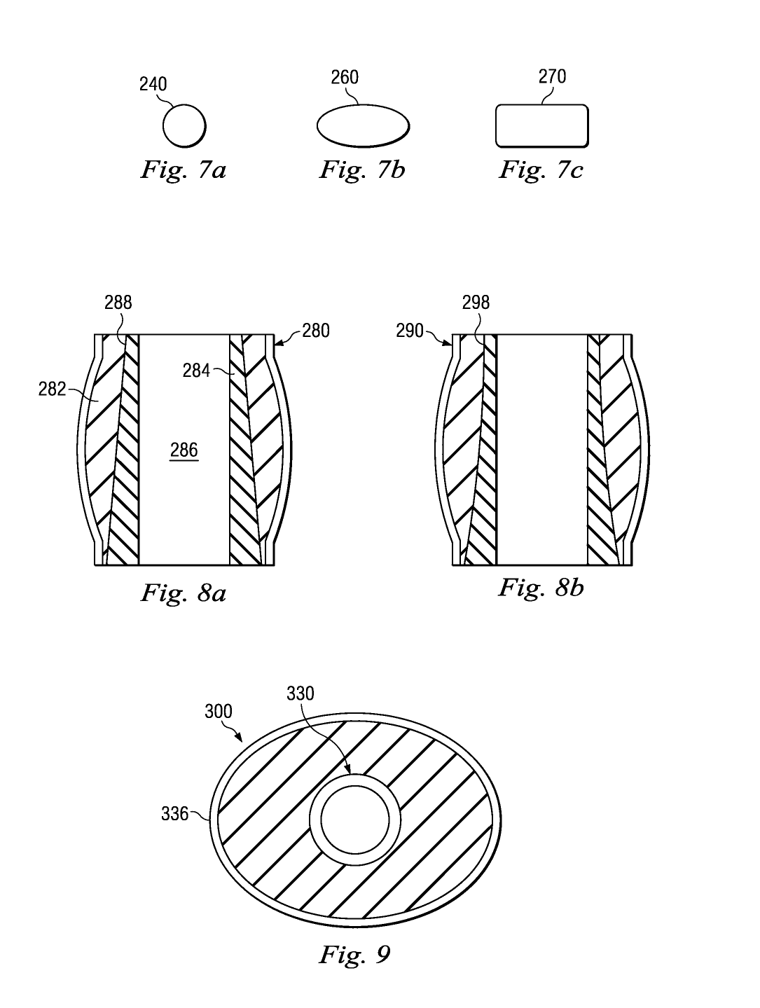

FIGS. 7a-7c are lateral cross-sectional views of various internal rod embodiments. In one embodiment, shown in FIG. 7a, internal rod 240 may be generally cylindrical and have similar, or isotropic properties in bending and in shear. In other embodiments, internal rod 240 may have anisotropic properties in bending and in shear. As shown in FIG. 7b, an exemplary internal rod 260 may have a generally oval cross-section. In this embodiment, internal rod 260 may provide for greater shear strength across a longer diameter, but with increased flexibility across a shorter diameter. For example, internal rod 260 may provide greater resistance to anterior-posterior bending by having the longer axis aligned anterior-posterior while at the same time having less resistance to lateral bending.

FIG. 7c shows a similar embodiment with an exemplary internal rod 270 having a generally rectangular cross-section. In this embodiment, internal rod 270 may provide for less shear strength across its width but with increased stiffness across its length. It is contemplated that in some embodiments, channel 232 is formed to have a profile shape matching the shape of the internal rods shown in FIGS. 7a-7c.

Internal rod 240 may be tuned to exhibit specific properties by changing materials, and/or by varying the cross-section. In yet another embodiment, internal rod 240 may have a continuous diameter or a variable diameter. A variable diameter internal rod may provide varying rigidity at some levels, or for more rigidity in extension and more flexibility in flexion. For example, FIG. 6a shows a cross-section of an exemplary device 200a, according to one embodiment. An internal rod 240a is shown having a varied diameter. In particular, it is shown that a core 230a maintains a consistent outer diameter while increasing in flexibility. This is because internal rod 240a transitions to a smaller diameter. A sleeve 238a may have a corresponding internal transition that decreases the internal diameter of sleeve 238a while the diameter of internal rod 240a is transitioning smaller. In addition, sleeve 238a and internal rod 240a may be comprised of different materials.

Thus, the cross-section of internal rod 240 and/or corresponding channel 232 may have any number of shapes in addition to those shown. Further, internal rod 240 may be modular, and a particular configuration may be selected by a surgeon based on pathology.

FIGS. 8a and 8b are longitudinal cross-sectional views of a toroidal damper 242 damper according to two exemplary embodiments. As shown in FIG. 8a, an exemplary damper 280 is shown comprised of at least two different portions 282 and 284, both comprised of elastomers with different durometers. The outer damper portion 282 may have a first elasticity that is less than a second elasticity used to construct the inner damper portion 284. Running through the center of inner damper portion 284 is a longitudinal passage 286 to accommodate core 230.

A transitioning interface 288 between inner and outer damper portions 282 and 284 may be linear as shown in FIG. 8a, or non-linear as shown by interface 298 in FIG. 8b. Interface 288, as an example, may allow for a mixed response to compression by the resilient damper such as a softer initial response that is followed by a stiffer final response—as compared to a resilient damper comprised of a homogeneous material. FIG. 8b shows a damper 290, according to one exemplary embodiment, comprised of two materials. Interface 298 has a non-linear interface that may offer an increased rate of change of elasticity—as compared to a damper comprised of a homogenous material.

Additional embodiments may include staggered, spiral, and other shaped transitions between inner and outer damper portions. In some embodiments the damper may generally take the form of a hollow cylinder (see, for example, damper 142, shown in FIGS. 2 and 3) or other shapes but still be comprised of more than one elastomer. Thus, by varying the transition area between inner and outer damper portions or by varying the elastomeric materials, the compressive force of the damper may be customized to provide a desired response while still maintaining the same general shape.

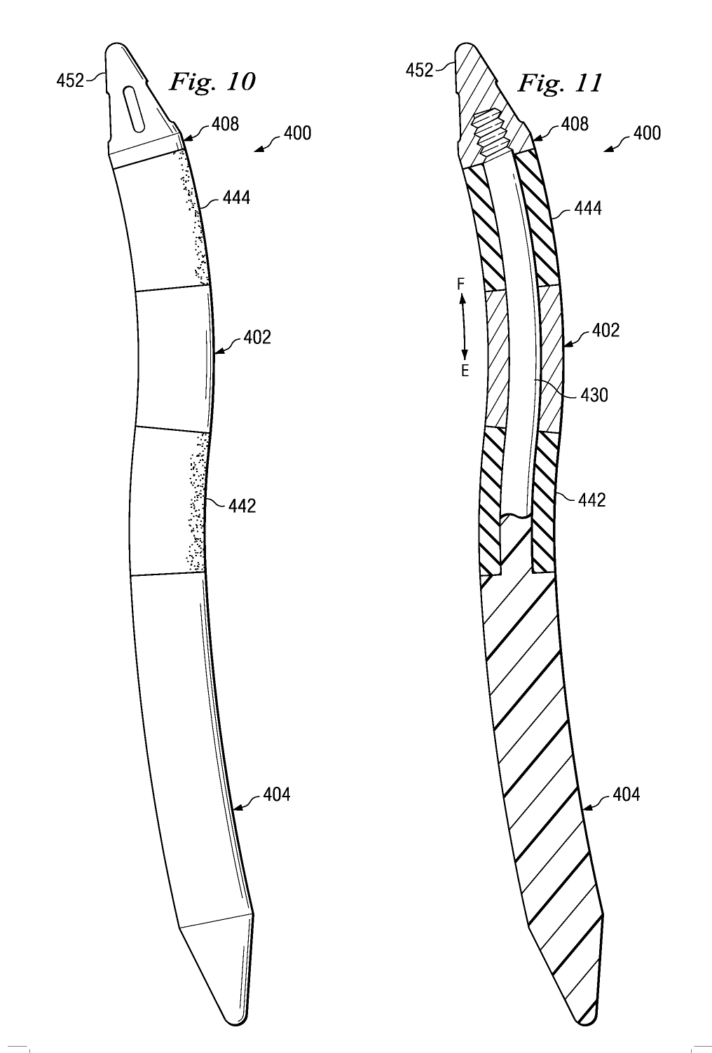

FIG. 9 is a lateral cross-section view of a damper according to another embodiment 300. As shown in FIG. 9, exemplary damper 300 is generally oval in cross-section with a first diameter greater than a second diameter. Damper 300 is surrounded by sheath 336. Damper 300 has a greater volume of resilient, or compressible material on either side of core 330 along the first diameter which may enable dampening against greater forces as compared to dampening capability of a smaller volume of compressible material on either side of core 330 along the second diameter. Thus, by changing the lateral cross-section of the damper, different functional properties may be obtained in different directions.

FIGS. 10 and 11 show an exemplary embodiment 400. FIG. 10 is a perspective view of a device 400 according to an exemplary embodiment. Device 400 has collar 402 that may be configured to the shape of a kinematic curve and which is slidably coupled to a base portion 404. FIG. 11 is a cross-section of device 400 and shows that base portion 404 is constructed as a monolith with a stem 430. Base portion 404 is configured to the lordotic curve. Stem 430 is at least partially configured to the shape of the kinematic curve and provides for slidable coupling with collar 402.

A first resilient damper 442 is disposed about stem 430 and is generally constrained between base portion 404 and the collar 402. A second resilient damper 444 is also disposed about stem 430 and is generally constrained between collar 402 and a cap 452. Cap 452 is attached at a superior end 408 of device 400. Cap 452 may be fixedly attached during assembly of device 400 or removably attached (as described with respect to cap portion 252 above).

As shown by motion arrows E-F in FIG. 11, collar 402 is able to slidably compress first resilient damper 442 when the spine is in extension and slidably compress second resilient damper 444 when the spine is in flexion.

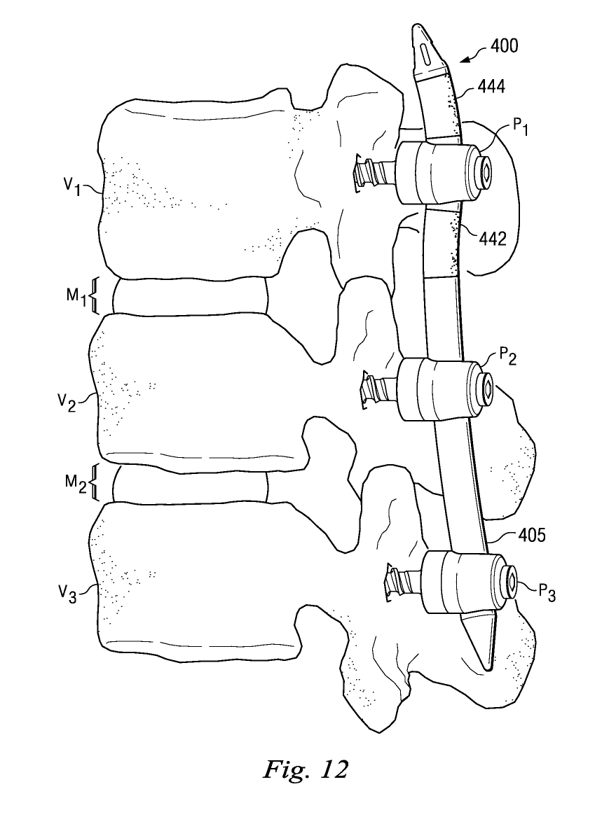

FIG. 12 is a side view of device 400 according to one exemplary embodiment installed across three vertebrae V₁, V₂ and V₃. As shown in FIG. 12, collar 402 is attached to first vertebra V₁ via pedicle screw P₁. Coupled to collar 402 is an extended length base portion 405, according to an extended embodiment that lengthens base portion 404 to extend between two vertebrae. Thus, base portion 405 extends between vertebrae V₂ and V₃, being attached to pedicle screws P₂ and P₃. Second resilient damper 444 is positioned superior to pedicle screw P₁ and first resilient damper 442 is positioned between adjacent vertebrae V₂ and V₃.

Accordingly, a V₁-V₂ motion segment M₁ is allowed to bend in flexion and extension. Motion segment M₁ is limited in flexion by compression of collar 402 against damper 444. Motion segment M₁ is limited in extension by compression of collar 402 against damper 442. A V₂-V₃ motion segment M₂ is substantially fixed against motion since base portion 405 is attached to pedicle screws P₂ and P₃. In yet other embodiments, device 400 maybe designed to function across only one spinal level. In other embodiments, two or more spinal levels may be treated with the devices disclosed herein. It is also contemplated that more or less dampers and collars and/or rod portions than disclosed herein may be used.

The constituent non-elastic, or non-resilient members may be formed of a suitable biocompatible material including, but not limited to, metals such as cobalt-chromium alloys, titanium alloys, nickel titanium alloys, aluminum, stainless steel alloys, and/or NITINOL or other memory alloy. In one embodiment, first and second end portions 102 and 104 and core 130 are formed of a cobalt-chrome-molybdenum metallic alloy (ASTM F-799 or F-75). Ceramic materials such as aluminum oxide or alumina, zirconium oxide or zirconium, compact of particulate diamond, and/or pyrolytic carbon may also be suitable.

Polymer materials may also be used alone or in combination with reinforcing elements, including polyetheretherketone (PEEK), polyethylene terephthalate (PET), polyester, polyetherketoneketone (PEKK), polylactic acid materials (PLA and PLDLA), polyaryletherketone (PAEK), carbon-reinforced PEEK, polysulfone, polyetherimide, polyimide, ultra-high molecular weight polyethylene (UHMWPE), cross-linked UHMWPE, and/or polycarbonate, among others. In one embodiment, first and second end portions 102 and 104 are formed of PEEK and core 130 is formed of titanium.

In some embodiments, different features, such as a second end sleeve and an internal core, are formed of dissimilar materials. In other embodiments, the entire second end portion and core are formed of a single material. Some materials may be selected for their particular properties. For example, a carbon nano-tube material may be selected for its excellent strength to size ratio or resistance to lateral shear forces, and reinforced polymers in general may be selected for their aniostropy.

In one embodiment an internal core may be constructed from a shape memory alloy with an s-shaped memory that is pliable at a first temperature for insertion into the s-shaped device, and becoming more rigid at a second temperature, such as body temperature. In another embodiment, the first and second end portions and the core are constructed from memory-alloy that may make the rigid portions of the device remain pliable for insertion into pedicle screws in misaligned vertebrae at a first temperature. After insertion, the s-shaped device seeks to return to its pre-formed kinematic and lordotic curvatures and becomes more rigid at a second temperature, thereby pulling the misaligned vertebrae into alignment with the pre-formed curvatures.

The bio-compatible sheath is made from fabric that is knitted, woven or braided in one embodiment, and may comprise a homogenous weave, or may comprise a fabric weave with anisotropic properties. In another embodiment, a sheath may be comprised of a non-woven, but flexible material. Whether woven or non-woven, the sheath may be formed from elastic, inelastic, semi-elastic material, or some combination of these or other materials. Exemplary inelastic materials which may be used for strands in the sheath are included in the list of inelastic materials above, but may particularly include titanium, memory-wire, ultra-high molecular weight polyethylene (UHMWPE), and/or cross-linked UHMWPE, among others.

Exemplary bio-compatible elastic materials which may be used for the resilient components include polyurethane, silicone, silicone-polyurethane, polyolefin rubbers, hydrogels, and the like. Other suitable elastic materials may include NITINOL or other superelastic alloys. Further, combinations of superelastic alloys and non-metal elastic materials may be suitable to form elastic strands. The elastic materials may be resorbable, semi-resorbable, or non-resorb able.

Multiple methods of accessing the surgical sight to accomplish the purposes of this disclosure are contemplated. In one embodiment, a posterior surgical approach is used. Pedicle screws are attached as known in the art and a novel device according to an exemplary embodiment in this disclosure is selected. The novel device is positioned, then secured to the pedicle screws.

In another embodiment, a kit may be provided to the surgeon comprising multiple components having varying properties, or multiple devices having varying properties. Thus, the surgeon may select an internal rod based material or cross-section from the kit based on a particular pathology or treatment strategy. Such a kit may also include an assortment of dampers of varying properties as discussed above, such as variable stiffness properties, varied cross-sections and varied wall thickness. In addition, a surgeon may measure or observe a patient's lordosis, thereby enabling the surgeon to select a device (or components) from the kit having the desired lordotic curve. The lordotic curve may also be modified by using a bending tool. Use of such a kit may also contemplate some assembly of an appropriate device by the surgeon.

Although device 100 has been illustrated and described as providing a specific combination of motion, it should be understood that other combinations of articulating movement are also possible and are contemplated as falling within the scope of the present invention, such as lateral bending and torsional bending.

In addition, correction of a spondylolisthesis defect as shown in FIG. 4 is an exemplary application of the disclosed embodiments. Other applications will be apparent to those skilled in the art and may include selective immobilization of the vertebral disc and/or the facet joints, motion-preservation of various motion segments and protective limiting of motion for weakened systems.

According to one embodiment, instruments and techniques for conducting a variety of surgical procedures are provided. In the illustrated embodiments, these procedures are conducted on the spine. However, the same devices and techniques may be used at other places in the body.

In addition, certain features and benefits are discussed with respect to certain embodiments. It is contemplated that any feature disclosed on any specific embodiment may be utilized on any other embodiment.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “cephalad,” “caudal,” “upper,” and “lower,” are for illustrative purposes only and may be varied within the scope of the disclosure. In the claims, means-plus-function clauses are intended to cover the elements described herein as performing the recited function and not only structural equivalents, but also equivalent elements.

We claim:

1. A motion-preserving spinal rod comprising:
an elongate first rod portion extending generally along a first curved path, the first rod portion having a distal end, a proximal end and an intermediate portion extending therebetween, wherein at least a portion of the first curved path substantially approximates a kinematic curve defined by flexion and extension of a superior vertebra relative to an inferior vertebra;
an elongate second rod portion coupled to the first rod portion, the second rod portion extending along a second curved path, the second rod portion having a distal end, a proximal end and an intermediate portion extending therebetween, wherein at least a portion of the second curved path substantially approximates a posterior lordotic curve, and wherein the first curved path is oriented relative to the second curved path to substantially form an S-shaped curve with the second curved path;
a core extending between the first rod portion and the second rod portion; and
a resilient damper disposed about the core, between the first and second rod portions, wherein the resilient damper is configured to provide resilient dampening of compressive force during vertebral extension.

2. The motion-preserving spinal rod of claim 1, further comprising:
a bio-compatible, flexible sheath having first and second ends, wherein the first end is attached to the distal end of the first rod portion and the second end is attached to the proximal end of the second rod portion, the sheath substantially surrounding the resilient damper, and wherein the sheath is configured to provide resistance in tension during vertebral flexion.

3. The motion-preserving spinal rod of claim 1, wherein the core is comprised of an internal rod received in a guide sleeve, the guide sleeve is integral with the second elongated end portion, and the first elongated end portion includes an internal elongate channel, the core being slidably inserted therein.

4. The motion-preserving spinal rod of claim 3, wherein one of the internal rod and the guide sleeve has a variable diameter and is configured to modify a stiffness of the core.

5. The motion-preserving spinal rod of claim 3, wherein the internal rod is comprised of a shape memory metal having a pliable condition at a first temperature and a rigid condition at a second temperature, whereby the internal rod is pliable for insertion into the first and second rod portions at the first temperature.

6. A motion-preserving spinal rod comprising:
an elongate first rod portion extending generally along a first curved path, the first rod portion having a distal end, a proximal end and an intermediate portion extending therebetween, wherein at least a portion of the first curved path substantially approximates a kinematic curve defined by flexion and extension of a superior vertebra relative to an inferior vertebra;
an elongate second rod portion coupled to the first rod portion, the second rod portion extending along a second curved path, the second rod portion having a distal end, a proximal end and an intermediate portion extending therebetween, wherein at least a portion of the second curved path substantially approximates a posterior lordotic curve, and wherein the first curved path is oriented relative to the second curved path to substantially form an S-shaped curve with the second curved path;
a core extending between the first rod portion and the second rod portion;
a damper disposed about the core, between the first and second rod portions, wherein the damper is configured to provide dampening of compressive force during vertebral extension; and
a sheath having first and second ends, the first end attached to the first rod portion and the second end attached to the second rod portion, the sheath substantially surrounding the variable stiffness damper, and wherein the sheath is configured to provide resilient dampening of tensile force during vertebral flexion and limitation of vertebral motion during flexion.

7. The motion-preserving spinal rod of claim 6, wherein the damper is a variable stiffness damper and is comprised of:
a first elastomer having a first durometer measurement;
a second elastomer bonded to the first elastomer, the second elastomer having a second durometer measurement different from the first durometer measurement; and
an interface where the first and second elastomers are bonded together.

8. The motion-preserving spinal rod of claim 7, wherein the interface between the first and second elastomers is one of linear and non-linear.

9. The motion-preserving spinal rod of claim 6, wherein the damper has a general toroidal shape with upper and lower ends with a middle area extending therebetween, the damper having a greater volume of resilient material around the middle area and a lesser volume of resilient material towards the upper and lower ends.

10. The motion-preserving spinal rod of claim 6, wherein the damper has an external surface and an internal bore, aligned along a longitudinal axis, the external surface and internal bore defining a wall thickness, the wall thickness being thicker in a first lateral direction and thinner in a second lateral direction, the damper providing greater compressive dampening in the first lateral direction than in the second lateral direction.

11. The motion-preserving spinal rod of claim 6, wherein the sheath has a slack condition during vertebral extension, a partially tensed condition during vertebral flexion, and a fully tensed condition at a maximum vertebral flexion, and wherein the sheath provides resilient dampening of tension in the partially tensed condition by exerting inward, radial force on the resilient damper and non-resilient tension resistance in the fully tensed condition.

12. A motion-preserving spinal rod comprising:
a generally S-shaped, elongate rod comprising a stem portion having at least one first diameter, the stem portion extending longitudinally from a base portion having at least one second diameter larger than the first diameter,
the stem portion extending at least partially along a first curved path, the stem portion having a first end and a second end, wherein the first curved path substantially approximates a kinematic curve generated by flexion and extension of adjacent superior and inferior vertebrae,
the base portion extending at least partially along a second curved path, the base portion having a first end and a second end, the base portion first end attached to the stem portion second end, wherein the second curved path substantially approximates a posterior lordotic curve, and wherein the first curved path is oriented relative to the second curved path to substantially form an S-shaped curve with the second curved path;
a collar slidingly disposed around the stem portion, the collar having a first end and a second end, the collar adapted to interface with a vertebral anchor;
a first resilient damper disposed about the stem portion and positioned between the base portion first end and the collar second end, wherein the first resilient damper is configured to provide resilient dampening of compressive force exerted by the collar during vertebral extension, the base portion first end configured to limit movement of the first resilient damper during vertebral extension; and
a retention member coupled to the stem portion first end.

13. The motion-preserving spinal rod of claim 12 further comprising:
a second resilient damper disposed about the stem portion and positioned between the collar first end and the retention member, wherein the second resilient damper is configured to provide resilient dampening of compressive force exerted by the collar during vertebral flexion, the retention member configured to limit movement of the second resilient damper during vertebral flexion.

14. The motion-preserving spinal rod of claim 13, wherein the first resilient member has a first outer diameter and the second resilient member has a second outer diameter, the first outer diameter being larger than the second outer such that the first resilient damper has a greater resilient capacity under compressive stress.

15. The motion-preserving spinal rod of claim 12, wherein the collar includes a longitudinal curvature substantially matching the first curved path.

16. The motion-preserving spinal rod of claim 13, wherein the retention member is releasably attached, and wherein one or more of the stem portion, the base portion, the first resilient damper, the second resilient damper and the collar are selectable from a kit, the kit comprising a plurality of at least one of stem portions, base portions, resilient dampers, and collars.

17. The motion-preserving spinal rod of claim 16, wherein the kit comprises a plurality of base portions, the base portions each having a different lordotic curvature.

18. The motion-preserving spinal rod of claim 16, wherein the kit comprises a plurality of stem portions, each of the plurality of stem portions having a kinematic radius of curvature, the plurality of stem portions including one or more stem portions with a kinematic radius of curvature selected from one of 30 mm, 45 mm, 50 mm, and 55 mm.

19. A method for stabilizing a spinal motion segment with a motion-preserving spinal rod comprising:
securing a first anchor to a first vertebra;
securing a second anchor to a second vertebra;
selecting a motion-preserving spinal rod, wherein the motion-preserving spinal rod comprises:
an elongate first rod portion extending generally along a first curved path, the first rod portion having a distal end, a proximal end and an intermediate portion extending therebetween, wherein at least a portion of the first curved path substantially approximates a kinematic curve defined by flexion and extension of a superior vertebra relative to an inferior vertebra;
an elongate second rod portion coupled to the first rod portion, the second rod portion extending generally along a second curved path, the second rod portion having a distal end, a proximal end and an intermediate portion extending therebetween, wherein at least a portion of the second curved path substantially approximates a posterior lordotic curve, and wherein the first curved path is oriented relative to the second curved path to substantially form an s-shaped curve with the second curved path;
a core extending between the first rod portion and the second rod portion; and
a resilient damper disposed about the core, between the first and second rod portions, wherein the resilient damper is configured to provide resilient dampening of compressive force during vertebral extension;
positioning the motion-preserving spinal rod between the first and second anchors; and
securing the motion-preserving spinal rod to the first and second anchors.

20. The method for stabilizing a spinal motion segment with a motion-preserving spinal rod of claim 19, wherein the first rod portion is configured to slide along the core, the first rod portion compressing the resilient damper during vertebral extension, the motion-preserving spinal rod further comprising:
a bio-compatible, flexible sheath having first and second ends, wherein the first end is attached to the distal end of the first rod portion and the second end is attached to the proximal end of the second rod portion, the sheath substantially surrounding the resilient damper, wherein the sheath is configured to prohibit motion of the first rod portion beyond a maximum position during vertebral flexion.

21. The method for stabilizing a spinal motion segment with a motion-preserving spinal rod of claim 19, wherein the proximal end of the first rod portion comprises a cap, the cap being removably attached to provide access to the core, wherein the core is removable and interchangeable.

22. The method for stabilizing a spinal motion segment with a motion-preserving spinal rod of claim 21, wherein selecting a motion-preserving spinal rod includes interchanging the core based on pathology.

23. The method for stabilizing a spinal motion segment with a motion-preserving spinal rod of claim 19, wherein selecting a motion-preserving spinal rod includes specifying a core configured to resist anterior-posterior shear forces between the first and second vertebrae.

24. The method for stabilizing a spinal motion segment with a motion-preserving spinal rod of claim 19, further comprising:
providing a surgical kit comprising a plurality of motion-preserving spinal rods with second rod portions configured according to different lordotic curvatures;
observing an actual posterior lordotic curve across the first and second vertebrae;
wherein the selecting a motion-preserving spinal rod is based on approximately matching a lordotic curvature of one of the plurality of second base portions to the actual posterior lordotic curve.

25. The method for stabilizing a spinal motion segment with a motion-preserving spinal rod of claim 19, further comprising:
securing a third anchor to a third vertebra;
wherein positioning the motion-preserving spinal rod includes positioning between the second and third anchors;
wherein securing the motion-preserving spinal rod includes securing to the third anchor; and
wherein the second rod portion is lengthened to be attachable between the second and third anchors, thereby preventing motion between the second and third vertebrae.
